(19) United States
(12) Patent Application Publication
(10) Pub. No.: US 2026/0345222 A1
(43) Pub. Date: Nov. 5, 2026

(54) ELECTROSURGICAL SYSTEMS AND METHODS INCORPORATING A JAW POSITION SENSOR TO FACILITATE TISSUE SEALING

(71) Applicant: Covidien LP, Mansfield, MA (US)

(72) Inventors: Robert F. MCCULLOUGH, JR., Boulder, CO (US); Hayden M. MAY, Westminster, CO (US); Jennifer L. RICH, Parker, CO (US); Kelley D. GOODMAN, Erie, CO (US); David D. BRAUSE, Longmont, CO (US); Caitlin K. YAMAKA, Westminster, CO (US); Jenna DANCY, Longmont, CO (US); Gregory W. FISCHVOGT, Reno, NV (US)

(73) Assignee: Covidien LP, Mansfield, MA (US)

(21) Appl. No.: 19/487,369

(22) PCT Filed: May 30, 2024
(86) PCT No.: PCT/IB2024/055292

§ 371 Date: Nov. 24, 2025

Related U.S. Application Data
(60) Provisional application No. 63/470,039, filed on May 31, 2023.

(51) Int. Cl. A61B 18/12; A61B 18/00; A61B 18/14

(52) U.S. Cl. CPC A61B 18/1206; A61B 18/1445; A61B 2018/0063; A61B 2018/00702; A61B 2018/00898

(57) ABSTRACT

An electrosurgical generator includes a processor and non-transitory computer-readable storage medium storing instructions that, when executed by the processor, cause the electrosurgical generator to supply electrosurgical energy to tissue grasped between first and second jaw members of an electrosurgical instrument according to a tissue sealing algorithm, determine whether the tissue sealing algorithm has been completed, determine a status of a sensor indicative of a position of the first and second jaw members, and determine whether the grasped tissue has been effectively sealed based on the determination of whether the tissue sealing algorithm has been completed and the determination of the status of the sensor.

This Application claims priority from U.S. Provisional Patent Application 63/470,039, filed 31 May 2023, the entire content of which is incorporated herein by reference.

### FIELD

The present disclosure relates to electrosurgery and, more particularly, to electrosurgical systems and methods incorporating a jaw position sensor to facilitate tissue sealing.

### BACKGROUND

Bipolar electrosurgery involves the conduction of electrical current through tissue positioned between electrodes of different polarity to heat and thereby treat the tissue. Bipolar electrosurgery often involves the use of an electrosurgical forceps, a pliers-like instrument that relies on mechanical action between its jaws to grasp, clamp, and constrict tissue. Electrosurgical forceps, more specifically, utilize mechanical clamping action and electrical energy to treat, e.g., cauterize, coagulate, and/or seal, clamped tissue.

Whereas cauterization involves the use of heat to destroy tissue and coagulation is a process of desiccating tissue such that the tissue cells are ruptured and dried, tissue sealing is a process of liquefying the collagen, elastin, and ground substances in the tissue so that they reform into a fused mass with significantly reduced demarcation between opposing tissue structures. In order to create an effective tissue seal, two predominant mechanical parameters are controlled: the pressure applied to the tissue and the gap distance between the electrodes. In addition, electrosurgical energy is applied to the tissue under controlled conditions, e.g., controlling the intensity, frequency, and/or duration of electrosurgical energy application to tissue, to ensure creation of an effective tissue seal.

### SUMMARY

As used herein, the term “distal” refers to the portion that is being described which is farther from an operator (whether a human user (surgeon, nurse, etc.) or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. Further, to the extent consistent, any or all of the aspects detailed herein may be used in conjunction with any or all of the other aspects detailed herein.

Provided in accordance with the present disclosure is an electrosurgical generator including a processor and non-transitory computer-readable storage medium storing instructions that, when executed by the processor, cause the electrosurgical generator to supply electrosurgical energy to tissue grasped between first and second jaw members of an electrosurgical instrument according to a tissue sealing algorithm, determine whether the tissue sealing algorithm has been completed, determine a status of a sensor indicative of a position of the first and second jaw members, and determine whether the grasped tissue has been effectively sealed based on the determination of whether the tissue sealing algorithm has been completed and the determination of the status of the sensor.

In an aspect of the present disclosure, the electrosurgical generator determines that the grasped tissue has been effectively sealed in response to determining that the tissue sealing algorithm has been completed and determining that the status of the sensor indicates that the first and second jaw members are disposed at or within a closure threshold.

In another aspect of the present disclosure, the electrosurgical generator is further caused to output a seal complete notification in response to determining that the grasped tissue has been effectively sealed.

In yet another aspect of the present disclosure, the electrosurgical generator is further caused to make a first determination that the grasped tissue has been effectively sealed in response to determining that the tissue sealing algorithm has been completed and determining that the status of the sensor indicates that the first and second jaw members are disposed at or within a closure threshold, and wherein the electrosurgical generator is further caused to make a second determination that the grasped tissue has been effectively sealed in response to determining that the tissue sealing algorithm has been completed and determining that the status of the sensor indicates that the first and second jaw members are disposed outside of the closure threshold.

In still another aspect of the present disclosure, the electrosurgical generator is further caused to output a first seal complete notification in response to the first determination that the grasped tissue has been effectively sealed and to output a second, different seal complete notification in response to the second determination that the grasped tissue has been effectively sealed.

In still yet another aspect of the present disclosure, the electrosurgical generator determines that the grasped tissue has not been effectively sealed in response to determining that the tissue sealing algorithm has not been completed.

In another aspect of the present disclosure, in response to determining that the grasped tissue has not been effectively sealed, the electrosurgical generator is further caused to output an error or supply electrosurgical energy to the tissue grasped between first and second jaw members according to an additional tissue sealing algorithm.

In still another aspect of the present disclosure, the electrosurgical generator determines that the grasped tissue has not been effectively sealed in response to determining that the tissue sealing algorithm has been completed and determining that the status of the sensor indicates that the first and second jaw members are disposed outside of the closure threshold.

In yet another aspect of the present disclosure, the electrosurgical generator determines that tissue sealing algorithm has been completed by determining that an end parameter of the tissue sealing algorithm has been reached.

Another electrosurgical generator provided in accordance with the present disclosure includes a processor and non-transitory computer-readable storage medium storing instructions that, when executed by the processor, cause the electrosurgical generator to determine whether an electrosurgical instrument has been activated, determine an initial status of a sensor indicative of a position of first and second jaw members of the electrosurgical instrument, and supply electrosurgical energy to tissue grasped between the first and second jaw members of the electrosurgical instrument based on the determination of whether the electrosurgical instrument has been activated and the determination of the initial status of the sensor.

In an aspect of the present disclosure, the electrosurgical generator is caused to supply the electrosurgical energy to the tissue grasped between the first and second jaw members according to a first tissue sealing algorithm in response to determining that the electrosurgical instrument has been activated and determining that the initial status of the sensor indicates that the first and second jaw members are disposed at or within a closure threshold, and supply the electrosurgical energy to the tissue grasped between the first and second jaw members according to a second, different tissue sealing algorithm in response to determining that the electrosurgical instrument has been activated and determining that the initial status of the sensor indicates that the first and second jaw members are disposed outside of the closure threshold.

In another aspect of the present disclosure, the electrosurgical generator is caused to output a warning prior to supplying the electrosurgical energy to the tissue grasped between the first and second jaw members in response to determining that the electrosurgical instrument has been activated and determining that the initial status of the sensor indicates that the first and second jaw members are disposed outside of a closure threshold and/or delay, for a pre-determined time period, supplying the electrosurgical energy to the tissue grasped between the first and second jaw members in response to determining that the electrosurgical instrument has been activated and determining that the initial status of the sensor indicates that the first and second jaw members are disposed outside of the closure threshold.

In still another aspect of the present disclosure, the electrosurgical generator is further caused to determine whether a tissue sealing algorithm associated with the supply of the electrosurgical energy to the grasped tissue is complete, determine a subsequent status of the sensor indicative of the position of the first and second jaw members, and determine whether the grasped tissue has been effectively sealed based on the determination of whether the tissue sealing algorithm has been completed and the determination of the subsequent status of the sensor.

In yet another aspect of the present disclosure, the initial status of the sensor is determined prior to the supply of the electrosurgical energy and the subsequent status of the sensor is determined after the supply of the electrosurgical energy.

In still yet another aspect of the present disclosure, the electrosurgical generator is further caused to inhibit the supply of the electrosurgical energy to the tissue grasped between the first and second jaw members of the electrosurgical instrument in response to determining that the electrosurgical instrument has been activated and determining that the initial status of the sensor indicates that the first and second jaw members are disposed outside of the closure threshold.

In another aspect of the present disclosure, the electrosurgical generator is further caused to require confirmation prior to supplying the electrosurgical energy to the tissue grasped between the first and second jaw members of the electrosurgical instrument in response to determining that the electrosurgical instrument has been activated and determining that the initial status of the sensor indicates that the first and second jaw members are disposed outside of the closure threshold.

An electrosurgical system provided in accordance with the present disclosure includes an end effector assembly including first and second jaw members at least one of which is movable relative to the other to grasp tissue therebetween, an activator configured to output an activator state indicative of whether the activator is un-activated or activated, a sensor configured to output a sensor state indicative of a position of the first and second jaw members, and an electrosurgical generator configured to supply electrosurgical energy to tissue grasped between the first and second jaw members in accordance with a tissue sealing algorithm to seal the grasped tissue. The electrosurgical generator includes sensor circuitry configured to monitor the grasped tissue. The electrosurgical generator is configured to determine whether the grasped tissue can be sealed based upon the output of the activator and the output of the sensor, determine whether the tissue sealing algorithm has been completed based upon the output of the sensor and the monitoring of the grasped tissue, and determine whether the tissue sealing algorithm has not been completed based upon the output of the sensor and the monitoring of the grasped tissue.

In an aspect of the present disclosure, the electrosurgical generator is configured to select the tissue sealing algorithm based on the output of the sensor.

In another aspect of the present disclosure, the sensor circuitry is configured to monitor an impedance of the grasped tissue.

In still another aspect of the present disclosure, the end effector assembly, the activator, and the sensor are incorporated into an electrosurgical forceps. The electrosurgical forceps is connected to the electrosurgical generator.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1 is a side view of an electrosurgical system in accordance with the present disclosure including an electrosurgical forceps and an electrosurgical generator;

FIG. 2 is a side, cutaway view of a proximal portion of the electrosurgical forceps of the system of FIG. 1;

FIG. 3 is a longitudinal, cross-sectional view taken along section line 3 -3 of FIG. 2 illustrating a jaw position sensor of the electrosurgical forceps disposed in an open condition;

FIG. 4 is a perspective view of an end effector assembly of the electrosurgical forceps with first and second jaw members of the end effector assembly disposed in an open configuration corresponding to the open condition of the sensor;

FIG. 5 is an enlarged, perspective view of a portion of the electrosurgical forceps during actuation of the end effector assembly from the open configuration towards a closed configuration with the sensor maintained in the open condition;

FIG. 6 is an enlarged, perspective view of a portion of the electrosurgical forceps during further actuation of the end effector assembly towards the closed configuration with the sensor disposed in a closed condition;

FIG. 7 is a side view of the end effector assembly of the electrosurgical forceps with the first and second jaw members in a partially closed configuration corresponding to the closed condition of the sensor;

FIG. 8 is a side view of the end effector assembly of the electrosurgical forceps with the first and second jaws in a further closed configuration;

FIG. 9 is a block diagram of the electrosurgical generator of the system of FIG. 1;

FIGS. 10-17 are flow diagrams of various methods of tissue sealing provided in accordance with the present disclosure; and

FIG. 18 is a schematic illustration of a robotic surgical system configured for use in accordance with aspects of the present disclosure.

### DETAILED DESCRIPTION

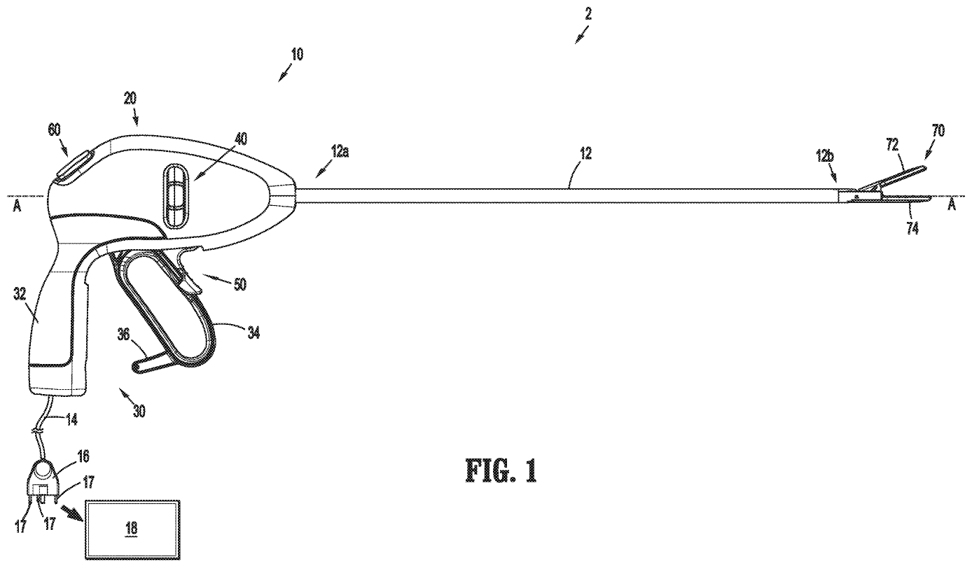

The present disclosure provides electrosurgical systems and methods incorporating a jaw position sensor to facilitate tissue sealing. Referring to FIG. 1, an electrosurgical system 2 provided in accordance with the present disclosure includes an electrosurgical forceps 10 and an electrosurgical generator 18. Forceps 10 is described to the extent necessary for the purposes herein without obscuring the present disclosure in unnecessary detail. Further aspects and features configured for use with forceps 10 are detailed, for example, in Patent Application Pub. No. US 2022/0183746, the entire contents of which are hereby incorporated herein by reference. Although described with respect to forceps 10, the aspects and features of the present disclosure are equally applicable for use with other suitable electrosurgical instruments including hemostat-style electrosurgical instruments, robotic electrosurgical instruments, etc.

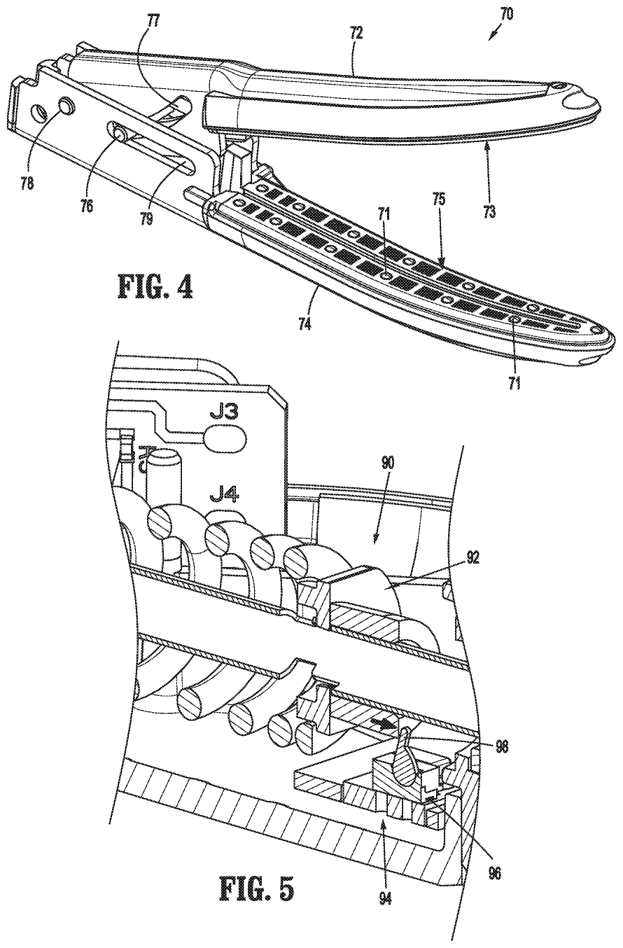

Continuing with reference to FIG. 1, forceps 10 includes a housing 20, a handle assembly 30, a rotating assembly 40, a trigger assembly 50, an activation assembly 60, and an end effector assembly 70. End effector assembly 70 includes first and second jaw members 72, 74, at least one of which is pivotable relative to the other about a pivot pin 78 (FIG. 4; or other suitable pivot structure) to grasp tissue to enable sealing and/or dividing of the grasped tissue. Forceps 10 further includes a shaft 12 that defines a longitudinal axis “A-A” and has a proximal end portion 12a operatively engaged to housing 20 and a distal end portion 12b operably engaged to end effector assembly 70. Forceps 10 also includes an electrosurgical cable 14 having a plug 16 configured to connect forceps 10 to generator 18. More specifically, plug 16 includes a plurality of pins 17 or other suitable contacts configured to mate with corresponding receptacles or other contacts of a port (not shown) of generator 18 to enable generator 18 to communicate with forceps 10 and control the supply of electrosurgical energy to end effector assembly 70 of forceps 10 for sealing tissue grasped between first and second jaw members 72, 74.

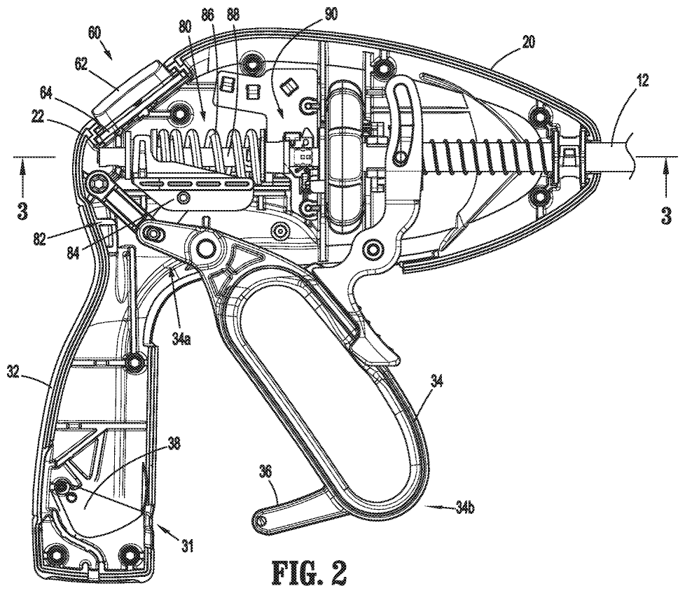

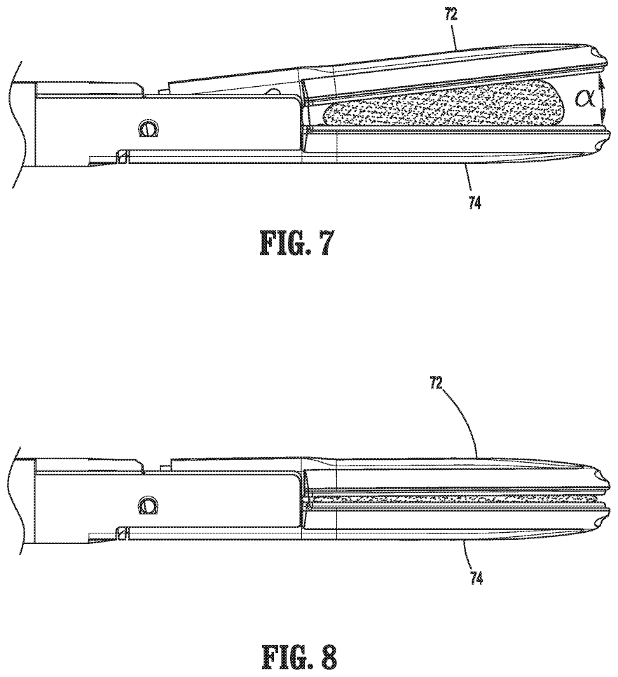

Referring to FIGS. 1 and 2, handle assembly 30 includes a fixed handle 32 and a movable handle 34. Fixed handle 32 is integrally associated with housing 20 and movable handle 34 is movable relative to the fixed handle 32 to actuate a drive assembly 80 of forceps 10. Movable handle 34 has an upper end portion 34a that is pivotally secured within housing 20 and operably engaged with a drive assembly 80 including a plurality of links 82, a carriage 84, a compression spring 86, and a drive tube 88. Upper end portion 34a of movable handle 34 operably couples to links 82 that cooperate to move carriage 84 distally against compression spring 86 in response to actuation of movable handle 34 towards fixed handle 32. Movement of carriage 84 distally against compression spring 86, in turn, regulates translation of drive tube 88 through shaft 12 and relative to end effector assembly 70 to grasp tissue between jaw members 72, 74 and regulate the closure force applied to tissue grasped between jaw members 72, 74. More specifically, with additional reference to FIG. 4, movement of carriage 84 distally against compression spring 86 initially urges compression spring 86 to translate distally to thereby translate drive tube 88 distally through shaft 12 to drive relative movement of cam pin 76 through cam slots 77, 79 defined within respective first and second jaw members 72, 74 to thereby urge jaw member 72 to pivot about pivot pin 78 and relative to jaw member 74 from an open configuration (FIG. 4) towards a closed configuration (FIGS. 7 and 8) to grasp tissue between first and second jaw members 72, 74 and apply a closure force to the grasped tissue. As an alternative to distal movement of drive tube 88 closing jaw members 72, 74, it is also contemplated that the opposite configuration be provided, e.g., wherein proximal movement of drive tube 88 closes jaw members 72, 74, or that other suitable closure mechanisms be provided, e.g., wherein rotation of a drive element closes jaw members 72, 74.

Upon reaching a threshold closure force applied to tissue grasped between first and second jaw members 72, 74, further movement of carriage 84 distally against compression spring 86, e.g., in response to further actuation of movable handle 34 towards fixed handle 32, compresses compression spring 86 rather than translating compression spring 86 distally (due to the resistive force applied by tissue inhibiting further closure of jaw members 72, 74) such that first and second jaw members 72, 74 are maintained in position grasping tissue therebetween. In this manner, the closure force applied to tissue grasped between jaw members 72, 74 is regulated to maintain a closure force or closure force within a closure force range.

In aspects, the closure force applied to the grasped tissue in the closed position of jaw members 72, 74 may be regulated such that the closure pressure, measured at a jaw centroid along the lengths of jaw members 72, 74, may be in a range of (or the jaw pressure range may be) from about 70 psi to about 130 psi; in other aspects from about 80 psi to about 120 psi; and, in still other aspects, from about 90 psi to about 110 psi.

Although end effector assembly 70 is described as a unilateral assembly, e.g., wherein second jaw member 74 is fixed relative to shaft 12 and first jaw member 72 is pivotable relative to second jaw member 74 and shaft 12, a bilateral assembly, e.g., wherein both first and second jaw members 72, 74 are pivotable relative to one another and shaft 12, is also contemplated. Further, in order to drive relative movement of cam pin 76 through cam slots 77, 79, drive tube 88 may be translationally fixed to cam pin 76 and shaft 12 translationally fixed to pivot pin 78 such that translation of drive tube 88 moves cam pin 76 relative to jaw members 72, 74 (and, thus, cam slots 77, 79) to thereby drive relative movement of cam pin 76 through cam slots 77, 79 to pivot first jaw member 72 towards second jaw member 74 to grasp tissue therebetween and apply a closure force to the grasped tissue. Alternatively, drive tube 88 may be translationally fixed to pivot pin 78 and shaft 12 translationally fixed to cam pin 76 such that translation of drive tube 88 moves jaw members 72, 74 (and, thus, cam slots 77, 79) relative to cam pin 76 to thereby drive relative movement of cam pin 76 through cam slots 77, 79 to pivot first jaw member 72 towards second jaw member 74 from the open configuration (FIG. 4) towards the closed configuration (FIGS. 7 and 8) to grasp tissue between first and second jaw members 72, 74 and apply the closure force to the grasped tissue.

With reference to FIG. 4, each jaw member 72, 74 of end effector assembly 70 includes an electrically conductive tissue contacting surface 73, 75, respectively. Jaw members 72, 74 are configured to grasp tissue between electrically conductive tissue contacting surface 73, 75 in the closed configuration thereof. Electrically conductive tissue contacting surfaces 73, 75 are adapted to connect to generator 18 (FIG. 1), e.g., via suitable electrical lead wires, electrically conductive structures, or combinations thereof extending through shaft 12, housing 20, and electrosurgical cable 14 to two of the pins 17 of plug 16 (FIG. 1), to enable energization of electrically conductive tissue contacting surface 73, 75 with electrosurgical, e.g., Radio Frequency (RF), energy at different potentials to enable the conduction of the electrosurgical energy between electrically conductive tissue contacting surface 73, 75 and through tissue grasped therebetween to seal the tissue.

Either or both jaw member 72, 74 may further include one or more stop members 71 (FIG. 4) disposed on or otherwise associated with either or both tissue-contacting surfaces 73, 75 to maintain a minimum gap distance or gap distance within a minimum gap distance range between tissue contacting surfaces 73, 75 when jaw members 72, 74 are disposed in a fully closed position, thus inhibiting electrical shorting. Stop member(s) 71 (FIG. 4) may be insulative, partly insulative, and/or electrically isolated from either or both tissue contacting surfaces 73, 75. In aspects, the minimum gap distance may be within or the minimum gap distance range may be from about 0.001 inches to about 0.010 inches; in other aspects from about 0.001 inches to about 0.008 inches; and, in still other aspects form about 0.001 inches to about 0.006 inches. Other suitable gap distances and ranges are also contemplated. The gap distance may be determined as the maximum gap distance between the tissue contacting surfaces 73, 75.

Referring back to FIGS. 1 and 2, movable handle 34 includes a flange 36 extending proximally from a lower end portion 34b of movable handle 34. Flange 36 is configured to extend through an aperture 31 defined within fixed handle 32 and ultimately engage a latch 38 within fixed handle 32 that is configured to selectively lock and unlock the fixed and movable handles 32, 34 relative to one another upon sufficient actuation of movable handle 34. Upon initial movement of flange 36 through aperture 31 to engage latch 38, in response to an initial actuation of movable handle 34 towards fixed handle 32, fixed and movable handles 32, 34 are lock relative to one another to thereby latch first and second jaw members 72, 74 in a closed configuration. Upon subsequent movement initial of flange 36 within aperture 31, in response to a subsequent actuation of movable handle 34 towards fixed handle 32, flange 36 is disengaged from latch 38 such that fixed and movable handles 32, 34 are unlocked permitting return of movable handle 34 towards its initial position and return of jaw member 72 towards the open configuration. In aspects, flange 36 and latch 38 are omitted and movable handle 34 is manually maintained in approximation with fixed handle 32 to thereby maintain first and second jaw members 72, 74 in the closed configuration.

Rotating assembly 40 is engaged with shaft 12 within housing 20 and extends outwardly from either side of housing to enables a user to manually control the orientation of shaft 12 and thus, end effector assembly 70, relative to housing 20. In aspects, rotating assembly 40 is infinitely rotatable in either direction about the longitudinal axis “A-A” to similarly rotate end effector assembly 70 relative to housing 20. Alternatively, rotating assembly 40 may have a defined range of motion.

Trigger assembly 50 enables a user to advance a knife (not shown) between first and second jaws 72, 74 to cut tissue grasped between jaw members 72, 74, e.g., once the grasped tissue is sealed. The knife may be configured for dynamically mechanically cutting tissue or may be energized (e.g., with electrosurgical energy) to dynamically electrically (or electromechanically) cut tissue grasped between jaw members 72, 74. As an alternative to a movable knife, a cutting electrode disposed within either or both jaw members 72, 74 may be provided for selective energization energized (e.g., with electrosurgical energy) to statically electrically (or electromechanically) cut tissue grasped between jaw members 72, 74. Other energy modalities for cutting tissue are also contemplated such as, for example, ultrasonic energy, thermal energy, light energy, microwave energy, etc.

Referring to FIGS. 1-3, activation assembly 60 is configured to signal generator 18 to initiate the supply of electrosurgical energy to first and second jaw members 72, 74 for sealing tissue. Activation assembly 60 includes an activation button 62 supported by a body 22 of housing 20. Activation button 62 is movable between an un-actuated position and an actuated position to thereby transition an underlying electrical switch 64 between a first state and a second state. Electrical switch 64, in turn, is adapted to electrically connect to generator 18, e.g., via one or more electrical lead wires extending from electrical switch 64 through housing 20 and electrosurgical cable 14 to one of the pins 17 (or other contact) of plug 16 to enable communication of the state of the electrical switch to generator 18. Generator 18, more specifically, may be configured to read an output of a corresponding pin 17, e.g., the presence of a resistance, voltage, current, etc. and/or a value of the resistance, voltage, current, etc., to detect the state of electrical switch 64 and, thus, to detect whether the user has activated activation button 62. For example, generator 18 may read the first state of electrical switch 64 as corresponding to an un-activated state and the second state of electrical switch 64 as corresponding to an activated state.

In aspects, activation button 62 is a two-stage actuator (as detailed above) and, thus, the output of electrical switch 64 read by generator 18 is binary, e.g., open or closed. Alternatively, activation button 62 may include three or more discrete stages or may be a continuous button such that electrical switch 64 includes three or more outputs capable of being read by generator 18. In such aspects, the outputs may include at least a first output corresponding to an un-activated state, a second output corresponding to a first activated state, and a third output corresponding to a second activated state. Other suitable activators configured to receive an input (e.g., a user input, input from another instrument, instructions based on feedback, etc.) and, in response thereto, to provide an output indicative of a state of the activator are also contemplated in place of activation button 62 and electrical switch 64 including, for example, other mechanical actuators and underlying switches, virtual buttons, icons on a touch-screen graphical user interface (GUI), pattern or gesture recognition devices, etc. Further, the activator need not be provided on forceps 10 but, rather, may be associated with generator 18 or remote from both forceps 10 and generator 18.

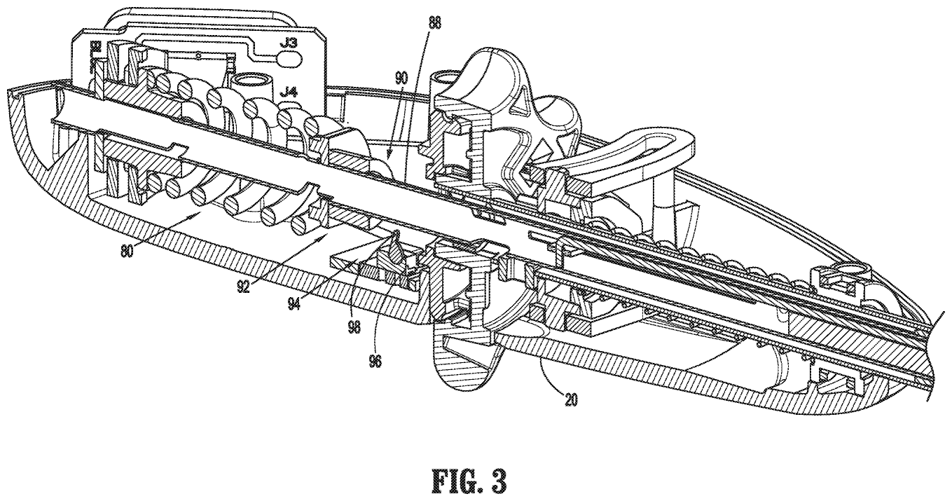

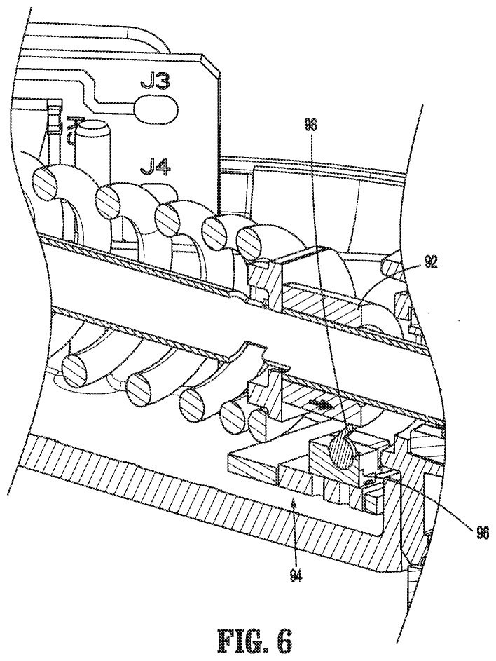

Turning to FIGS. 3-8, forceps 10 includes a jaw position sensor assembly 90. Sensor assembly 90 is utilized to determine a state of closure of first and second jaw members 72, 74 relative to one another to thereby detect whether jaw members 72, 74 are adequately approximated and applying an adequate closure force to tissue grasped between jaw members 72, 74 for effective tissue sealing. Sensor assembly 90 includes a mechanical flag 92 associated with drive tube 88 and a sensor 94 associated with housing 20, although the reverse configuration is also contemplated. Sensor 94 includes a mechanical toggle 98 and an underlying electrical switch 96. Mechanical flag 92 is fixed relative to drive tube 88 of drive assembly 80 such that, when drive tube 88 is moved a predetermined distance corresponding to the first and second jaw members 72, 74 being closed to or beyond a pre-determined jaw aperture angle “α,” mechanical flag 92 is urged into contact with mechanical toggle 98 of sensor 94 to thereby actuate mechanical toggle 98 to change a state of underlying electrical switch 96 from an open condition to a closed condition (or vice versa). That is, flag 92 is configured to contact and move toggle 98 to change the state of electrical switch 96 at the pre-determined jaw aperture angle “α.” Prior to jaw members 72, 74 reaching this pre-determined jaw aperture angle “α,” drive tube 88 and flag 92 are positioned proximally of sensor 94 and, thus, electrical switch 96 is maintained in its at-rest, open condition. Thus, an open configuration of first and second jaw members 72, 74, e.g., wherein jaw members 72, 74 are open at an angle greater than the pre-determined jaw aperture angle “α,” corresponds to an open state of switch 96 and a closed configuration of first and second jaw members 72, 74, e.g., wherein jaw members 72, 74 are closed at an angle equal to or less than the pre-determined jaw aperture angle “α,” corresponds to a closed state of switch 96, although the opposite configuration is also contemplated.

Electrical switch 96 of sensor 94 is adapted to electrically connect to generator 18 (FIG. 1), e.g., via one or more electrical lead wires extending from electrical switch 96 through housing 20 and electrosurgical cable 14 to one of the pins 17 (or other contact) of plug 16 to enable communication of the state of electrical switch 96 to generator 18 (see FIG. 1). Electrical switch 96 of sensor 94 may share a pin 17 (or other contact) of plug 16 with switch 64 of activation assembly 60 (see FIGS. 1 and 2) or may include a separate pin 17 (FIG. 1) or other contact. In either configuration, generator 18 (FIG. 1) is configured to read an output of the corresponding pin 17 (FIG. 1), e.g., the presence of a resistance, voltage, current, etc. and/or a value of the resistance, voltage, current, etc., to detect the state of electrical switch 96 and, thus, to detect whether jaw members 72, 74 are open at an angle above the pre-determined jaw aperture angle “α” or closed at an angle equal to or less than the pre-determined jaw aperture angle “α.”

In aspects, toggle 98 is a two-stage actuator and, thus, the output of electrical switch 96 read by generator 18 (FIG. 1) is binary, e.g., open or closed. Alternatively, toggle 98 may include three or more discrete stages or may be a continuous toggle such that electrical switch 96 includes three or more outputs capable of being read by generator 18 (FIG. 1), e.g., open, partially closed (including multiple ranges of partial closure), fully closed, etc.

Although an exemplary sensor assembly 90 is detailed above, other suitable sensor assemblies for determining whether jaw members 72, 74 are open at an angle above the pre-determined jaw aperture angle “α” or closed at an angle equal to or less than the pre-determined jaw aperture angle “α” are also contemplated. For example, distance sensors, proximity sensors, and/or surgical cameras may be utilized to enable determination of the angle of jaw members 72, 74 relative to the pre-determined jaw aperture angle “α” such as, for example, as disclosed in: U.S. Pat. No. 8,357,158 titled “JAW CLOSURE DETECTION SYSTEM;” U.S. Pat. No. 10,695,123 titled “SURGICAL INSTRUMENT WITH SENSOR;” or Patent Application Publication No. US 2017/0215944, titled “JAW APERTURE POSITION SENSOR FOR ELECTROSURGICAL FORCEPS,” the entire contents of each of which are hereby incorporated herein by reference.

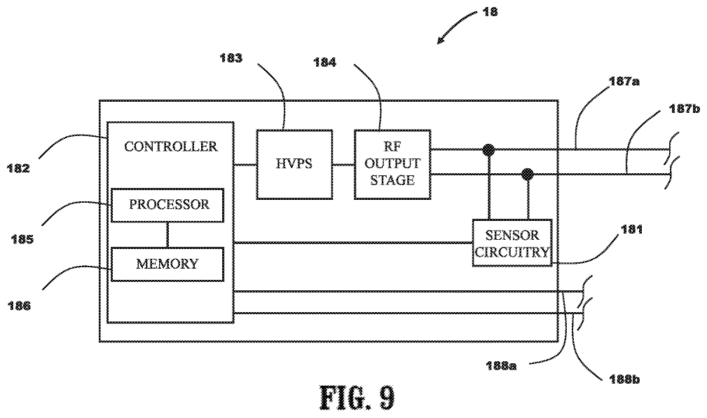

Referring to FIG. 9, generator 18 includes sensor circuitry 181, a controller 182, a high voltage DC power supply (“HVPS”) 183 and an RF output stage 184. HVPS 183 provides high voltage DC power to RF output stage 184 which converts the high voltage DC power into RF electrosurgical energy for delivery to end effector assembly 70 (FIG. 4), e.g., tissue-contacting surfaces 73, 75 of jaw members 72, 74, respectively, of end effector assembly 70 (see FIG. 4). In particular, RF output stage 184 may generate sinusoidal waveforms of high frequency RF energy. RF output stage 184 may be configured to generate a plurality of waveforms having various duty cycles, peak voltages, crest factors, and other parameters, depending on a particular mode of operation and/or electrical feedback data.

Controller 182 includes a processor 185 (e.g., a microprocessor) operably connected to a non-transitory computer-readable storage medium such as a memory 186 which may be volatile type memory (e.g., RAM) and/or non-volatile type memory (e.g., flash media, disk media, etc.). Processor 185 is operably connected to HVPS 183 and/or RF output stage 184 allowing processor 185 to control the output of generator 18. Sensor circuitry 181 is operably coupled between RF output stage 184 and electrical leads 187a, 187b, which connect to receptacle contacts (not shown) of generator 18 that are configured to interface with corresponding pins 17 of plug 16 (FIG. 1) to supply energy to/from tissue-contacting surfaces 73, 75 of respective jaw members 72, 74 (FIG. 4). Sensor circuitry 181 may thus determine one or more parameters, e.g., tissue impedance, output current, and/or voltage, etc. associated with the supply of energy to tissue. Sensor circuitry 181 provides feedback, e.g., based on the sensed parameter(s), to controller 182 which, in turn, selects an energy-delivery algorithm, modifies an energy-delivery algorithm, adjusts energy-delivery parameters, and/or determines a status of an energy-delivery algorithm based thereon. Controller 182 may also monitor the signal provided by electrical lead 188a (which connects to a receptacle contact(s) (not shown) of generator 18 that is configured to interface with a corresponding pin 17 of plug 16 (FIG. 1) to communicate with activation assembly 60 (FIGS. 1-2)) to determine the state of activation assembly 60, e.g., to determine whether activation button 62 is in the un-actuated position or the actuated position, and electrical lead 188b (which connects to a receptacle contact(s) (not shown) of generator 18 that is configured to interface with a corresponding pin 17 of plug 16 (FIG. 1) to communicate with sensor assembly 90 (FIGS. 3 and 5)) to determine the state of the electrical switch of sensor 90, e.g., to determine whether jaw members 72, 74 are open at an angle above the pre-determined jaw aperture angle “α” or closed at an angle equal to or less than the pre-determined jaw aperture angle “a.”

As demonstrated below, controller 182 may be configured to utilize the state of activation assembly 60, the state of sensor assembly 90 (before initiation of a tissue sealing algorithm and/or after completion of a tissue sealing algorithm), and/or feedback from sensor circuitry 181 to enable initiation of tissue sealing, selection and implementation of a tissue sealing algorithm(s), and/or determination of whether tissue sealing has been successfully completed.

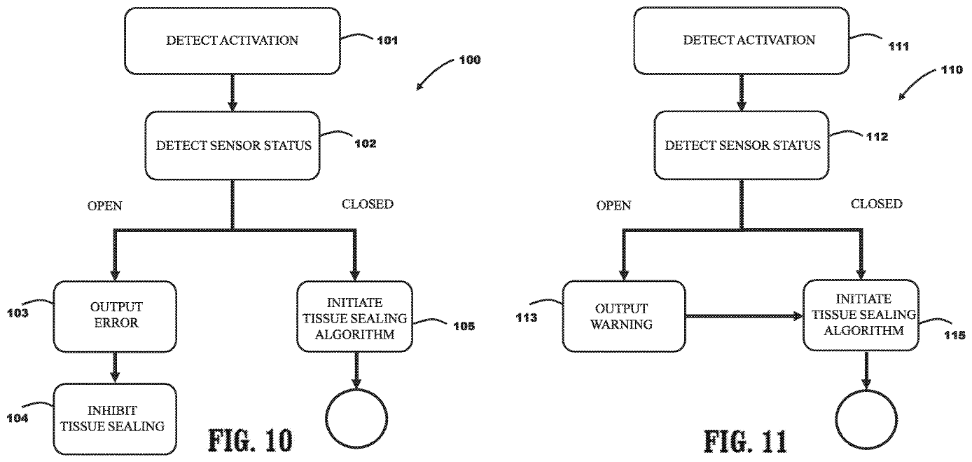

Turning to FIGS. 10-17, in conjunction with FIGS. 1-9, methods of tissue sealing using electrosurgical system 2 configured to be executed by controller 182 of generator 18 of electrosurgical system 2 and leveraging the output of sensor assembly 90 are described. Although described with respect to electrosurgical system 2, controller 182 of generator 18, and sensor assembly 90, methods 100-170 of FIGS. 10-17 are also contemplated for use with any other suitable electrosurgical systems and/or components thereof.

Utilizing the output of sensor assembly 90 (or other suitable sensor assembly to indicate jaw position before and/or after implementing a tissue sealing algorithm) together with electrosurgical feedback (to determine completion of an implemented tissue sealing algorithm) to determine whether to proceed with tissue sealing and/or to evaluate the outcome of tissue sealing (and, in aspects, take appropriate action based upon the evaluated outcome) takes into account both the mechanical and electrical parameters that contribute to formation of an effective tissue seal, thus facilitating formation of effective tissue seals, providing warnings where effective tissue seals may not or potentially have not been established, and/or taking appropriate actions in response to determining that an effective tissue seal may not or potentially has not been established.

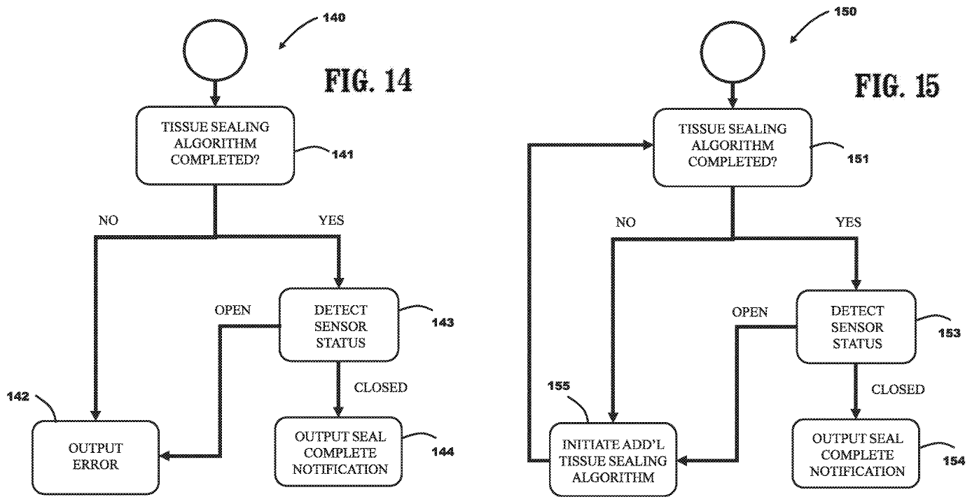

FIGS. 10-13 provide methods beginning at activation and proceeding to initiating (or not initiating) a tissue sealing algorithm, while FIGS. 14-17 provide methods for determining whether a tissue seal has been successfully created and, in aspects, providing a response where it is determined that a tissue seal may not have been successfully created. Thus, any of the methods of FIGS. 10-13 may be utilized with any of the methods of FIGS. 14-17 to provide a method from activation to seal completion (or determination of a potentially incomplete seal).

Referring to FIG. 10, in conjunction with FIGS. 1-9, method 100 begins with the detection of activation at 101, e.g., via generator 18 reading the state of activation assembly 60 as the second state corresponding to the activated position of activation button 62. That is, at 101, generator 18 may determine that a user has activated activation button 62, thus indicating that the user intends to initiate tissue sealing. However, activation as detected at 101 need not be user-initiated but, rather, may be automatically initiated in response to a detected condition, e.g., grasping of tissue between jaw members 72, 74, at the direction of another instrument or system (e.g., a robotic system), or in any other suitable manner. Although any suitable activations and/or detections thereof are contemplated, method 100 is not initiated until activation is detected at 101.

Once activation is detected at 101, method 100 proceeds to 102 wherein the status of the sensor, e.g., sensor assembly 90, is detected. For example, in order to detect the status of the sensor at 102, generator 18 may read the state of electrical switch 96 of sensor assembly 90 corresponding to whether jaw members 72, 74 are open at an angle above the pre-determined jaw aperture angle “α” or closed at an angle equal to or less than the pre-determined jaw aperture angle “α.”

If the status of the sensor detected at 102 is “OPEN” or otherwise indicating that jaw members 72, 74 are open at an angle above the pre-determined jaw aperture angle “α,” method 100 proceeds to 103 where an error is output. The error output at 103 may be an audible tone, an audible message, illumination of one or more LED's, a notification (symbol, banner, illustration, and/or text) represented on a GUI, a haptic output, combinations thereof, or any other suitable error output to signal to a user that tissue sealing will not proceed. Generator 18, forceps 10, and/or other component of the system (e.g., a robotic console, communication hub, connected display, etc.) may include a speaker, one or more LED's, a GUI, a haptic device, and/or other suitable output device to produce the error output as instructed by generator 18 at 103. The error output at 103 may indicate that tissue sealing will not proceed, may also provide a rationale (e.g., that tissue sealing will not proceed because thick tissue is grasped between jaw members 72, 74 and/or jaw members 72, 74 are open at an angle above the pre-determined jaw aperture angle “α”), may provide a recommendation to the user (e.g., to re-grasp tissue and re-initiate tissue sealing), or may provide any other suitable indication of an error and/or that tissue sealing will not proceed.

After providing the output at 103, before providing the output at 103, simultaneously with providing the output at 103, or in place of providing the output at 103, generator 18 inhibits tissue sealing at 104, e.g., inhibiting the supply of energy to jaw members 72, 74. The supply of energy to jaw members 72, 74 may be inhibited until method 100 is restarted, e.g., upon detection of a subsequent activation at 101.

On the other hand, if the status of the sensor detected at 102 is “CLOSED” or otherwise indicating that jaw members 72, 74 are closed at an angle equal to or less than the pre-determined jaw aperture angle “α,” method 100 proceeds to 105 wherein generator 18 initiates the supply of energy to jaw members 72, 74 in accordance with a tissue sealing algorithm. The tissue sealing algorithm or portions thereof may be based on tissue impedance feedback (e.g., controlling power or other parameters to follow a target impedance profile); voltage-based control (e.g., constant voltage or following a voltage profile); current-based control (e.g., constant current or following a current profile); power-based control (e.g., constant power or following a power profile); combinations thereof; or any other suitable tissue sealing algorithm. Exemplary tissue sealing algorithms suitable for use in accordance with the present disclosure can be found in, for example, U.S. Pat. No. 8,920,421 titled “SYSTEM AND METHOD FOR TISSUE SEALING” or U.S. Pat. No. 8,147,485 titled “SYSTEM AND METHOD FOR TISSUE SEALING,” the entire contents of each of which are hereby incorporated herein by reference.

With reference to FIG. 11, in conjunction with FIGS. 1-9, method 110 begins with the detection of activation at 111, similarly as detailed above with respect to detecting activation at 101 of method 100 (FIG. 10). Once activation is detected at 111, method 110 proceeds to 112 wherein the status of the sensor is detected, similarly as detailed above with respect to detecting the status of the sensor at 102 of method 100 (FIG. 10).

If the status of the sensor detected at 112 is “OPEN” or otherwise indicating that jaw members 72, 74 are open at an angle above the pre-determined jaw aperture angle “α,” method 110 proceeds to 113 where a warning is output. The warning output at 113 may be an audible tone, an audible message, illumination of one or more LED's, a notification (symbol, banner, illustration, and/or text) represented on a graphical user interface, a haptic output, combinations thereof, or any other suitable output to signal to a user that thick tissue is grasped between jaw members 72, 74, that jaw members 72, 74 are open at an angle above the pre-determined jaw aperture angle “α,” and/or that tissue sealing may not be successful.

After providing the output at 113, the method proceeds to 115. In aspects, generator 18 is configured to implement a pre-determined delay period between providing the output at 113 and proceeding to 115. Such a delay may be, for example, 5 seconds, 10 seconds, or any other suitable delay sufficient to enable a user to abort the tissue sealing attempt in response to the warning output at 113 and prior to initiation of the supply of energy to jaw members 72, 74.

After the output at 113 or, if the status of the sensor detected at 112 is “CLOSED” or otherwise indicating that jaw members 72, 74 are closed at an angle equal to or less than the pre-determined jaw aperture angle “α,” method 110 proceeds to 115 wherein generator 18 initiates the supply of energy to jaw members 72, 74 in accordance with a tissue sealing algorithm, similarly as detailed above with respect to 105 of method 100 (FIG. 10).

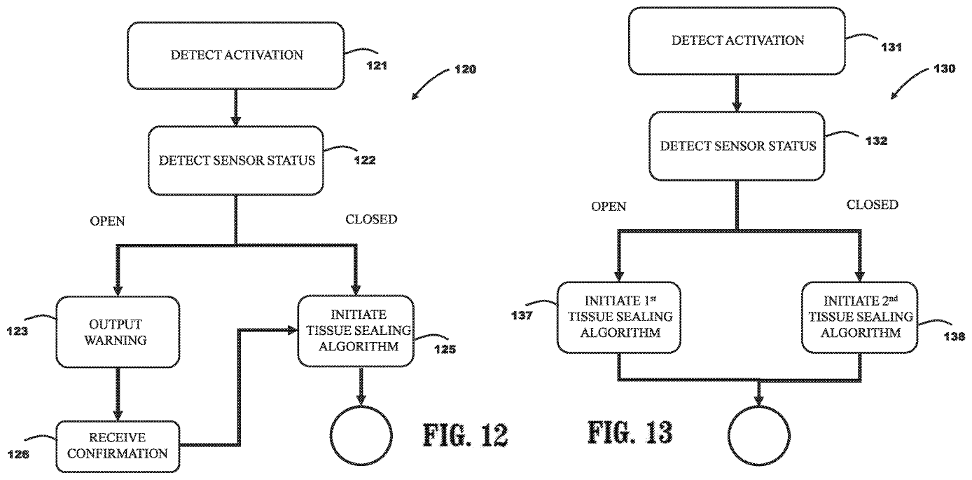

Turning to FIG. 12, in conjunction with FIGS. 1-9, method 120 is similar to method 110 (FIG. 11) in that method 120 begins with the detection of activation at 121 and, once activation is detected at 121, proceeds to 122 wherein the status of the sensor is detected.

If the status of the sensor detected at 122 is “OPEN” or otherwise indicating that jaw members 72, 74 are open at an angle above the pre-determined jaw aperture angle “α,” method 120 proceeds to 123 where a warning is output, similarly as detailed above with respect to method 110 (FIG. 11). However, rather than proceeding automatically to initiate tissue sealing after outputting the warning as in method 110 (FIG. 11), method 120 requires receipt of confirmation at 126 after output of the warning at 123 and prior to proceeding to 125. Confirmation at 126 may be in the form of any suitable user input such as, for example, a re-activation of activation button 62, selecting an appropriate icon on a GUI of generator 18 (or otherwise activating a button associated with forceps 10 and/or generator 18), depressing a footswitch or other actuator, providing a verbal command, etc. Confirmation at 126 helps ensure that the user has acknowledged the warning output at 123 and would still like to proceed. If no confirmation is received, e.g., within a pre-determined time period after output of the warning at 123, supply of energy to jaw members 72, 74 may be inhibited until method 120 is restarted, e.g., upon detection of a subsequent activation at 121.

If confirmation is received at 126, method 120, as noted above, proceeds to 125. After the confirmation at 126 or, if the status of the sensor detected at 122 is “CLOSED” or otherwise indicating that jaw members 72, 74 are closed at an angle equal to or less than the pre-determined jaw aperture angle “α,” method 120 proceeds to 125 wherein generator 18 initiates the supply of energy to jaw members 72, 74 in accordance with a tissue sealing algorithm, similarly as detailed above with respect to 105 of method 100 (FIG. 10).

Referring to FIG. 13, in conjunction with FIGS. 1-9, method 130 begins, similarly as detailed above with respect to methods 100-120 (FIGS. 10-12), with the detection of activation at 131 and, once activation is detected at 131, proceeds to 132 wherein the status of the sensor is detected.

If the status of the sensor detected at 132 is “OPEN” or otherwise indicating that jaw members 72, 74 are open at an angle above the pre-determined jaw aperture angle “α,” method 120 proceeds to 137 wherein generator 18 initiates the supply of energy to jaw members 72, 74 in accordance with a first tissue sealing algorithm. If, on the other hand, the status of the sensor detected at 132 is “CLOSED” or otherwise indicating that jaw members 72, 74 are closed at an angle equal to or less than the pre-determined jaw aperture angle “α,” method 130 proceeds to 138 wherein generator 18 initiates the supply of energy to jaw members 72, 74 in accordance with a second, different tissue sealing algorithm. The first and/or second tissue sealing algorithms may be configured similar to any of the tissue sealing algorithms detailed above and may differ from one another in duration, addition or subtraction of one or more portions of the tissue sealing algorithm, method of control (e.g., power, current, voltage, impedance), and/or in any other suitable manner. In aspects, the first tissue sealing algorithm includes a pre-treatment portion (while the second tissue sealing algorithm does not include a pre-treatment portion). A suitable pre-treatment portion for this purpose is described, for example, in Patent Application Pub. No. US 2022/0249154 titled “ELECTROSURGICAL SYSTEMS AND METHODS FOR SEALING TISSUE,” the entire contents of which are hereby incorporated herein by reference. Post-treatment portions for inclusion in the first tissue sealing algorithm (while the second tissue sealing algorithm does not include a post-treatment portion) are also contemplated, as are other suitable differences between the first and second tissue sealing algorithms.

In aspects, a notification output is provided before proceeding to 137 or 138 to indicate to the user that jaw members 72, 74 are open at an angle above the pre-determined jaw aperture angle “α” and/or that the first tissue sealing algorithm is to be implemented (e.g., prior to proceeding to 137) or to indicate to the user that jaw members 72, 74 are closed at an angle equal to or less than the pre-determined jaw aperture angle “α” and/or that the second tissue sealing algorithm is implemented (e.g., prior to proceeding to 138), respectively.

FIGS. 14-17, as noted above, provide methods for determining whether a tissue seal has been successfully created and, in aspects, providing a response where it is determined that a tissue seal may not have been successfully created. The methods of FIGS. 14-17 may thus be implemented after initiating a tissue sealing algorithm at 105 (FIGS. 10), 115 (FIGS. 11), 125 (FIGS. 12), 137 (FIG. 13), or 138 (FIG. 13). Alternatively, the methods of FIGS. 14-17 may be implemented after initiating a tissue sealing algorithm in any other suitable manner such as, for example, wherein the tissue sealing algorithm is initiated in response to detecting activation (e.g., as detailed above) without consideration of the status of the sensor.

Turning to FIG. 14, in conjunction with FIGS. 1-9, method 140 begins after initiating a tissue sealing algorithm, e.g., in accordance with any of methods 100-130 or any other suitable initiation of a tissue sealing algorithm. Method 140 initially includes, at 141, generator 18 determining whether the tissue sealing algorithm has been completed. Determining whether the tissue sealing algorithm has been completed may include, for example, determining that no error conditions were detected and/or determining that an end condition has been satisfied, e.g., that an end tissue impedance has been reached. Other suitable methods for determining whether the tissue sealing algorithm has been completed are also contemplated, including those detailed in U.S. Pat. No. 8,920,421; U.S. Pat. No. 8,147,485; and Patent Application Pub. No. US 2022/0249154, each of which was previously incorporated by reference herein.

If it is determined that the tissue sealing algorithm was not completed at 141 (“NO” at 141), method 140 proceeds to 142 wherein an error output is provided indicating that the tissue sealing algorithm was not completed. The error output may be provided in any of the manners detailed above.

If, on the other hand, it is determined that the tissue sealing algorithm was completed at 141 (“YES” at 141), method 140 proceeds to 143 wherein the status of the sensor is detected, similarly as detailed above with respect to methods 100-130 (FIGS. 10-13) except that checking the status of the sensor at 143 is performed after determining that the tissue sealing algorithm was completed (not prior to initiating tissue sealing as in methods 100-130 (FIGS. 10-13)).

If the status of the sensor detected at 143 is “OPEN” or otherwise indicating that jaw members 72, 74 are open at an angle above the pre-determined jaw aperture angle “α,” method 140 proceeds to 142 where an error is output. The error may be provided in any of the manners detailed above and may be the same error as provided when it is determined that the tissue sealing algorithm was not completed or may be a different error to distinguish the two situations.

If, on the other hand, the status of the sensor detected at 143 is “CLOSED” or otherwise indicating that jaw members 72, 74 are closed at an angle equal to or less than the pre-determined jaw aperture angle “α,” method 140 proceeds to 144 wherein a seal complete notification is output. The seal complete notification may include an audible tone, an audible message, illumination of one or more LED's, a notification (symbol, banner, illustration, and/or text) represented on a graphical user interface (GUI), a haptic output, combinations thereof, or any other suitable notification.

FIG. 15 illustrates a method 150 that begins with generator 18 (FIG. 1) determining whether the tissue sealing algorithm has been completed at 151, similarly as with method 140 (FIG. 14). If it is determined that the tissue sealing algorithm was not completed at 151 (“NO” at 151), method 150 proceeds to 155 wherein an additional tissue sealing algorithm is initiated. The additional tissue sealing algorithm may include re-applying the tissue sealing algorithm previously implemented, applying a different tissue sealing algorithm (such as, for example, a post-treatment portion of a tissue sealing algorithm), or any other suitable tissue sealing algorithm. After implementing the additional tissue sealing algorithm at 155, method 150 returns to 151 to determine whether the additional tissue sealing algorithm was completed.

If it is determined that the tissue sealing algorithm was completed at 151 (“YES” at 151), method 150 proceeds to 153 wherein the status of the sensor is detected, similarly as detailed above with respect to method 140 (FIG. 14). If the status of the sensor detected at 153 is “OPEN” or otherwise indicating that jaw members 72, 74 are open at an angle above the pre-determined jaw aperture angle “α” (see FIG. 7), method 150 proceeds to 155 where an additional tissue sealing algorithm is output as detailed above. The additional tissue sealing algorithm implemented in response to determining that the prior tissue sealing algorithm was not completed may be similar or different from the additional tissue sealing algorithm implemented in response to determining that the status of the sensor detected is “OPEN.”

If, on the other hand, the status of the sensor detected at 153 is “CLOSED” or otherwise indicating that jaw members 72, 74 are closed at an angle equal to or less than the pre-determined jaw aperture angle “α” (see FIG. 7), method 150 proceeds to 154 wherein a seal complete notification is output, as detailed above.

With reference to FIG. 16, method 160 begins with generator 18 (FIG. 1) determining whether the tissue sealing algorithm has been completed at 161, similarly as with methods 140, 150 (FIGS. 14 and 15, respectively). If it is determined that the tissue sealing algorithm was not completed at 161, method 160 may proceed similarly as detailed above with respect to method 140 (FIG. 14) or method 150 (FIG. 15). If it is determined that the tissue sealing algorithm was completed at 161 (“YES” at 161), method 160 proceeds to 163 wherein the status of the sensor is detected, similarly as detailed above with respect to methods 140, 150 (FIGS. 14 and 15, respectively). If the status of the sensor detected at 163 is “OPEN” or otherwise indicating that jaw members 72, 74 are open at an angle above the pre-determined jaw aperture angle “α” (see FIG. 7), method 160 proceeds to 166 where a first seal complete notification is output. If, on the other hand, the status of the sensor detected at 163 is “CLOSED” or otherwise indicating that jaw members 72, 74 are closed at an angle equal to or less than the pre-determined jaw aperture angle “α” (see FIG. 7), method 160 proceeds to 167 wherein a second seal complete notification is output. The first and second seal complete notifications may be provided in any of the manners detailed above and may be differentiated from one another to notify the user that, although the tissue sealing algorithm was completed in both instances, the first seal complete notification corresponds to a condition wherein the jaw members 72, 74 are open at an angle above the pre-determined jaw aperture angle “α” (see FIG. 7) at the completion of the tissue sealing algorithm while the second seal complete notification corresponds to a condition wherein jaw members 72, 74 are closed at an angle equal to or less than the pre-determined jaw aperture angle “α” (see FIG. 7) at the completion of the tissue sealing algorithm.

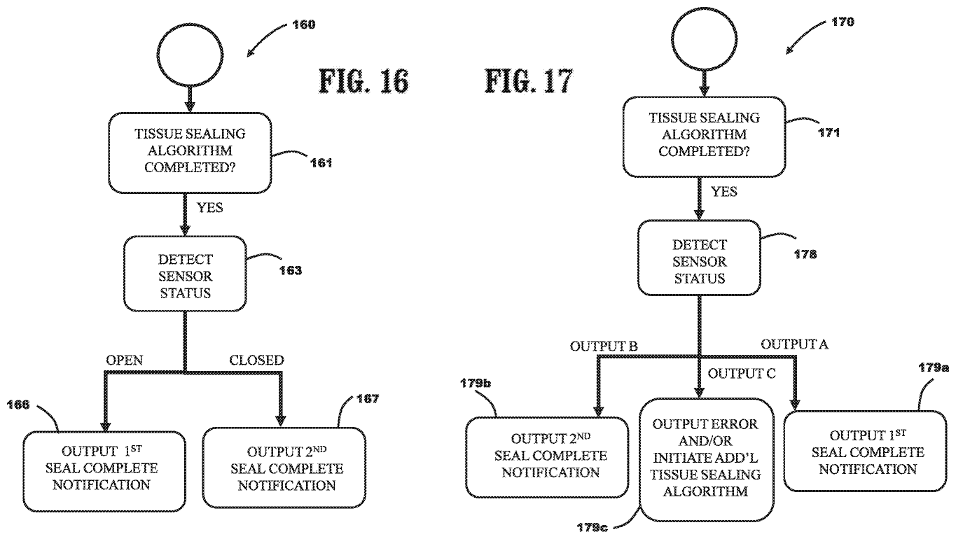

FIG. 17 illustrates method 170 which is similar to method 160 (FIG. 16), except as detailed below, and begins with generator 18 (FIG. 1) determining whether the tissue sealing algorithm has been completed at 171. If it is determined that the tissue sealing algorithm was completed at 171 (“YES” at 171), method 170 proceeds to 178 wherein the status of the sensor is detected. With respect to method 170, the sensor status includes three or more different statuses, e.g., a first output A, a second output B, and a third output C. Such may be the case where, for example, electrical switch 96 of sensor 94 (FIGS. 3, 5, and 6) includes three or more outputs capable of being read by generator 18 (FIG. 1), e.g., corresponding to a first state wherein jaw members 72, 74 (FIG. 4) closed at an angle equal to or less than a first threshold, corresponding to a second state wherein jaw members 72, 74 (FIG. 4) are partially closed at an angle greater than the first threshold but equal to or less than a second threshold, and corresponding to a third state wherein jaw members 72, 74 (FIG. 4) are open at an angle greater than the second threshold.

If the sensor status detected at 178 corresponds to the first state wherein jaw members 72, 74 (FIG. 4) are closed at an angle equal to or less than the first threshold, the method proceeds to 179a wherein a first seal complete notification is output. If the sensor status detected at 178 corresponds to the second state wherein jaw members 72, 74 (FIG. 4) are partially closed at an angle greater than the first threshold but equal to or less than a second threshold, the method proceeds to 179b wherein a second seal complete notification is output. The first and second seal complete notifications may be differentiated from one another similarly as detailed above with respect to method 160 (FIG. 16).

If, however, the sensor status detected at 178 corresponds to the third state wherein jaw members 72, 74 (FIG. 4) are open at an angle greater than the second threshold, the method proceeds to 179c, wherein an error is output (e.g., as detailed above with respect to 142 of method 140 (FIG. 14)) and/or an additional tissue sealing algorithm is initiated (e.g., as detailed above with respect to 155 of method 150 (FIG. 15)).

While aspects and features of the present disclosure are illustrated and described above as being utilized with a hand-held electrosurgical forceps, it is also contemplated that the aspects and features of the present disclosure be utilized with other surgical instruments and systems, such as robotic surgical systems and what is commonly referred to as “Telesurgery.” Such systems employ various robotic elements to assist the user and allow remote operation (or partial remote operation) of surgical instrumentation. Various robotic arms, gears, cams, pulleys, electric and mechanical motors, etc. may be employed for this purpose and may be designed with a robotic surgical system to assist the user during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to an operating theater or located in a remote location. In this instance, one team of users may prep a patient for surgery and configure the robotic surgical system with one or more of the instruments disclosed herein while another user (or group of user) remotely control the instruments via the robotic surgical system. As can be appreciated, a highly skilled user may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the user to produce a corresponding movement of the working ends of any type of surgical instrument (e.g., end effectors, graspers, knifes, scissors, etc.) which may complement the use of one or more of the aspects described herein. The movement of the master handles may be scaled so that the working ends have a corresponding movement that is different, smaller or larger, than the movement performed by the operating hands of the user. The scale factor or gearing ratio may be adjustable so that the operator can control the resolution of the working ends of the surgical instrument(s).

The master handles may include various sensors to provide feedback to the user relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the user with enhanced tactile feedback simulating actual operating conditions. The master handles may also include a variety of different actuators for delicate tissue manipulation or treatment further enhancing the user's ability to mimic actual operating conditions.

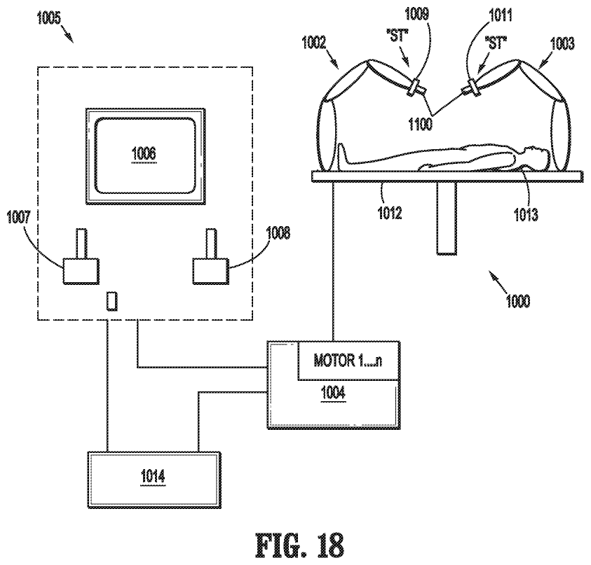

Referring now to FIG. 18, a medical workstation of a robotic surgical system such as detailed above is shown generally as workstation 1000 and generally includes a plurality of robot arms 1002, 1003; a control device 1004; and an operating console 1005 coupled with the control device 1004. The operating console 1005 may include a display device 1006, which may be set up in particular to display three-dimensional images; and manual input devices 1007, 1008, by means of which a user may be able to telemanipulate the robot arms 1002, 1003 in a first operating mode.

Each of the robot arms 1002, 1003 may include a plurality of members, which are connected through joints, and an attaching device 1009, 1011, to which may be attached, for example, a surgical tool “ST” supporting an end effector 1100. The surgical tool “ST” and end effector 1100 may include, for example, the above-detailed features of electrosurgical forceps 10 (FIG. 1) adapted for use in a robotic surgical system.

The robot arms 1002, 1003 may be driven by electric drives (not shown) that are connected to the control device 1004. The control device 1004 (e.g., a computer) may be set up to activate the drives, in particular by means of a computer program, in such a way that the robot arms 1002, 1003, their attaching devices 1009, 1011 and thus the surgical tool “ST” (including end effector 1100) execute a desired movement according to a movement defined by means of the manual input devices 1007, 1008. The control device 1004 may also be set up in such a way that it regulates the movement of the robot arms 1002, 1003 and/or of the drives.

The medical workstation 1000 may be configured for use on a patient 1013 lying on a patient table 1012 to be treated in a minimally invasive manner by means of the end effector 1100. The medical workstation 1000 may also include more than two robot arms 1002, 1003, the additional robot arms likewise being connected to the control device 1004 and being telemanipulatable by means of the operating console 1005. A medical instrument or surgical tool (including an end effector 1100) may also be attached to the additional robot arm. Medical workstation 1000 may include a database 1014, in particular coupled to the control device 1004, in which are stored, for example, pre-operative data from the patient/living being 1013 and/or anatomical atlases.

Aspects of this disclosure may be further described by reference to the following numbered paragraphs:
1. An electrosurgical generator including a processor and non-transitory computer-readable storage medium storing instructions that, when executed by the processor, cause the electrosurgical generator to: supply electrosurgical energy to tissue grasped between first and second jaw members of an electrosurgical instrument according to a tissue sealing algorithm; determine whether the tissue sealing algorithm has been completed; determine a status of a sensor indicative of a position of the first and second jaw members; and determine whether the grasped tissue has been effectively sealed based on the determination of whether the tissue sealing algorithm has been completed and the determination of the status of the sensor.
2. The electrosurgical generator according to paragraph 1, wherein the electrosurgical generator determines that the grasped tissue has been effectively sealed in response to determining that the tissue sealing algorithm has been completed and determining that the status of the sensor indicates that the first and second jaw members are disposed at or within a closure threshold.
3. The electrosurgical generator according to paragraph 2, wherein the electrosurgical generator is further caused to output a seal complete notification in response to determining that the grasped tissue has been effectively sealed.
4. The electrosurgical generator according to any one of paragraphs 1-3, wherein the electrosurgical generator is further caused to make a first determination that the grasped tissue has been effectively sealed in response to determining that the tissue sealing algorithm has been completed and determining that the status of the sensor indicates that the first and second jaw members are disposed at or within a closure threshold, and wherein the electrosurgical generator is further caused to make a second determination that the grasped tissue has been effectively sealed in response to determining that the tissue sealing algorithm has been completed and determining that the status of the sensor indicates that the first and second jaw members are disposed outside of the closure threshold.
5. The electrosurgical generator according to paragraph 4, wherein the electrosurgical generator is further caused to output a first seal complete notification in response to the first determination that the grasped tissue has been effectively sealed and to output a second, different seal complete notification in response to the second determination that the grasped tissue has been effectively sealed.
6. The electrosurgical generator according to any one of paragraphs 1-5, wherein the electrosurgical generator determines that the grasped tissue has not been effectively sealed in response to determining that the tissue sealing algorithm has not been completed.
7. The electrosurgical generator according to paragraph 6, wherein, in response to determining that the grasped tissue has not been effectively sealed, the electrosurgical generator is further caused to: output an error; or supply electrosurgical energy to the tissue grasped between first and second jaw members according to an additional tissue sealing algorithm.
8. The electrosurgical generator according to any one of paragraphs 1-7, wherein the electrosurgical generator determines that the grasped tissue has not been effectively sealed in response to determining that the tissue sealing algorithm has been completed and determining that the status of the sensor indicates that the first and second jaw members are disposed outside of the closure threshold.
9. The electrosurgical generator according to any one of paragraphs 1-8, wherein the electrosurgical generator determines that tissue sealing algorithm has been completed by determining that an end parameter of the tissue sealing algorithm grasped tissue has been reached.
10. An electrosurgical generator including a processor and non-transitory computer-readable storage medium storing instructions that, when executed by the processor, cause the electrosurgical generator to: determine whether an electrosurgical instrument has been activated; determine an initial status of a sensor indicative of a position of first and second jaw members of the electrosurgical instrument; and supply electrosurgical energy to tissue grasped between the first and second jaw members of the electrosurgical instrument based on the determination of whether the electrosurgical instrument has been activated and the determination of the initial status of the sensor.
11. The electrosurgical generator according to paragraph 10, wherein the electrosurgical generator is caused to: supply the electrosurgical energy to the tissue grasped between the first and second jaw members according to a first tissue sealing algorithm in response to determining that the electrosurgical instrument has been activated and determining that the initial status of the sensor indicates that the first and second jaw members are disposed at or within a closure threshold; and supply the electrosurgical energy to the tissue grasped between the first and second jaw members according to a second, different tissue sealing algorithm in response to determining that the electrosurgical instrument has been activated and determining that the initial status of the sensor indicates that the first and second jaw members are disposed outside of the closure threshold.
12. The electrosurgical generator according to paragraphs 10 or 11, wherein the electrosurgical generator is caused to at least one of: output a warning prior to supplying the electrosurgical energy to the tissue grasped between the first and second jaw members in response to determining that the electrosurgical instrument has been activated and determining that the initial status of the sensor indicates that the first and second jaw members are disposed outside of a closure threshold; delay, for a pre-determined time period, supplying the electrosurgical energy to the tissue grasped between the first and second jaw members in response to determining that the electrosurgical instrument has been activated and determining that the initial status of the sensor indicates that the first and second jaw members are disposed outside of the closure threshold.
13. The electrosurgical generator according to any one of paragraphs 10-12, wherein the electrosurgical generator is further caused to: determine whether a tissue sealing algorithm associated with the supply of the electrosurgical energy to the grasped tissue is complete; determine a subsequent status of the sensor indicative of the position of the first and second jaw members; and determine whether the grasped tissue has been effectively sealed based on the determination of whether the tissue sealing algorithm has been completed and the determination of the subsequent status of the sensor.
14. The electrosurgical generator according to paragraph 13, wherein the initial status of the sensor is determined prior to the supply of the electrosurgical energy and wherein the subsequent status of the sensor is determined after the supply of the electrosurgical energy.
15. The electrosurgical generator according to any one of paragraphs 10-14, wherein the electrosurgical generator is further caused to inhibit the supply of the electrosurgical energy to the tissue grasped between the first and second jaw members of the electrosurgical instrument in response to determining that the electrosurgical instrument has been activated and determining that the initial status of the sensor indicates that the first and second jaw members are disposed outside of the closure threshold.
16. The electrosurgical generator according to any one of paragraphs 10-15, wherein the electrosurgical generator is further caused to require confirmation prior to supplying the electrosurgical energy to the tissue grasped between the first and second jaw members of the electrosurgical instrument in response to determining that the electrosurgical instrument has been activated and determining that the initial status of the sensor indicates that the first and second jaw members are disposed outside of the closure threshold.
17. An electrosurgical system, comprising: an end effector assembly including first and second jaw members, at least one of the first or second jaw members movable relative to the other to grasp tissue therebetween, an activator configured to output an activator state indicative of whether the activator is un-activated or activated; a sensor configured to output a sensor state indicative of a position of the first and second jaw members; and an electrosurgical generator configured to supply electrosurgical energy to tissue grasped between the first and second jaw members in accordance with a tissue sealing algorithm to seal the grasped tissue, the electrosurgical generator including sensor circuitry configured to monitor the grasped tissue, wherein the electrosurgical generator is configured to determine whether the grasped tissue can be sealed based upon the output of the activator and the output of the sensor, determine whether the tissue sealing algorithm has been completed based upon the output of the sensor and the monitoring of the grasped tissue, and determine whether the tissue sealing algorithm has not been completed based upon the output of the sensor and the monitoring of the grasped tissue.
18. The electrosurgical system according to paragraph 17, wherein the electrosurgical generator is configured to select the tissue sealing algorithm based on the output of the sensor.
19. The electrosurgical system according to paragraph 17 or 18, wherein the sensor circuitry is configured to monitor an impedance of the grasped tissue.
20. The electrosurgical system according to any one of paragraphs 17-19, wherein the end effector assembly, the activator, and the sensor are incorporated into an electrosurgical forceps, the electrosurgical forceps connected to the electrosurgical generator.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular configurations. Those skilled in the art will envision other modifications within the scope and spirit of the claims appended hereto.

1. An electrosurgical generator including a processor and non-transitory computer-readable storage medium storing instructions that, when executed by the processor, cause the electrosurgical generator to:
supply electrosurgical energy to tissue grasped between first and second jaw members of an electrosurgical instrument according to a tissue sealing algorithm;
determine whether the tissue sealing algorithm has been completed;
determine a status of a sensor indicative of a position of the first and second jaw members and
determine whether the grasped tissue has been effectively sealed based on the determination of whether the tissue sealing algorithm has been completed and the determination of the status of the sensor.

2. The electrosurgical generator according to claim 1, wherein the electrosurgical generator determines that the grasped tissue has been effectively sealed in response to determining that the tissue sealing algorithm has been completed and determining that the status of the sensor indicates that the first and second jaw members are disposed at or within a closure threshold.

3. The electrosurgical generator according to claim 2, wherein the electrosurgical generator is further caused to output a seal complete notification in response to determining that the grasped tissue has been effectively sealed.

4. The electrosurgical generator according to claim 1, wherein the electrosurgical generator is further caused to make a first determination that the grasped tissue has been effectively sealed in response to determining that the tissue sealing algorithm has been completed and determining that the status of the sensor indicates that the first and second jaw members are disposed at or within a closure threshold.

5. The electrosurgical generator according to claim 15, wherein the electrosurgical generator is further caused to output a first seal complete notification in response to the first determination that the grasped tissue has been effectively sealed and to output a second, different seal complete notification in response to the second determination that the grasped tissue has been effectively sealed.

6. The electrosurgical generator according to claim 1, wherein the electrosurgical generator determines that the grasped tissue has not been effectively sealed in response to determining that the tissue sealing algorithm has not been completed.

7. The electrosurgical generator according to claim 6, wherein, in response to determining that the grasped tissue has not been effectively sealed, the electrosurgical generator is further caused to one of:
output an error; or
supply electrosurgical energy to the tissue grasped between first and second jaw members according to an additional tissue sealing algorithm.

8. The electrosurgical generator according to claim 1, wherein the electrosurgical generator determines that the grasped tissue has not been effectively sealed in response to determining that the tissue sealing algorithm has been completed and determining that the status of the sensor indicates that the first and second jaw members are disposed outside of the closure threshold.

9. The electrosurgical generator according to claim 1, wherein the electrosurgical generator determines that tissue sealing algorithm has been completed by determining that an end parameter of the tissue sealing algorithm has been reached.

10. The electrosurgical generator according to claim 1, wherein the electrosurgical generator is further caused to:
determine whether the electrosurgical instrument has been activated;
determine an initial status of the sensor indicative of the position of first and second jaw members of the electrosurgical instrument; and
supply the electrosurgical energy to the tissue grasped between the first and second jaw members based on the determination of whether the electrosurgical instrument has been activated and the determination of the initial status of the sensor.

11. The electrosurgical generator according to claim 10, wherein the electrosurgical generator is further caused to:
supply the electrosurgical energy to the tissue grasped between the first and second jaw members according to a first tissue sealing algorithm in response to determining that the electrosurgical instrument has been activated and determining that the initial status of the sensor indicates that the first and second jaw members are disposed at or within a closure threshold; and
supply the electrosurgical energy to the tissue grasped between the first and second jaw members according to a second, different tissue sealing algorithm in response to determining that the electrosurgical instrument has been activated and determining that the initial status of the sensor indicates that the first and second jaw members are disposed outside of the closure threshold.

12. The electrosurgical generator according to claim 10, wherein the electrosurgical generator is further caused to output a warning prior to supplying the electrosurgical energy to the tissue grasped between the first and second jaw members in response to determining that the electrosurgical instrument has been activated and determining that the initial status of the sensor indicates that the first and second jaw members are disposed outside of a closure threshold.

13. The electrosurgical generator according to claim 10, wherein the electrosurgical generator is further caused to inhibit the supply of the electrosurgical energy to the tissue grasped between the first and second jaw members of the electrosurgical instrument in response to determining that the electrosurgical instrument has been activated and determining that the initial status of the sensor indicates that the first and second jaw members are disposed outside of the closure threshold.

14. The electrosurgical generator according to claim 10, wherein the electrosurgical generator is further caused to require confirmation prior to supplying the electrosurgical energy to the tissue grasped between the first and second jaw members of the electrosurgical instrument in response to determining that the electrosurgical instrument has been activated and determining that the initial status of the sensor indicates that the first and second jaw members are disposed outside of the closure threshold.

15. The electrosurgical generator according to claim 4, wherein the electrosurgical generator is further caused to make a second determination that the grasped tissue has been effectively sealed in response to determining that the tissue sealing algorithm has been completed and determining that the status of the sensor indicates that the first and second jaw members are disposed outside of the closure threshold.

16. The electrosurgical generator according to claim 10, wherein the electrosurgical generator is further caused to delay, for a pre-determined time period, supplying the electrosurgical energy to the tissue grasped between the first and second jaw members in response to determining that the electrosurgical instrument has been activated and determining that the initial status of the sensor indicates that the first and second jaw members are disposed outside of the closure threshold.
